(19) United States
(12) Patent Application Publication
(10) Pub. No.: US 2012/0199703 A1
(43) Pub. Date: Aug. 9, 2012

(54) Spiral Wire Retaining Device and Method of Using the Same

(76) Inventor: Kevin D. Taylor, Colorado Springs, CO (US)

(21) Appl. No.: 13/367,210

(22) Filed: Feb. 6, 2012

Related U.S. Application Data
(60) Provisional application No. 61/440,245, filed on Feb. 7, 2011.

(51) Int. Cl. A61B 19/00; F16L 3/08

(52) U.S. Cl. 248/70; 248/299.1

(57) ABSTRACT

A retaining device for retaining at least one medical line adjacent to a supporting surface comprising a spiral-shaped holding portion defining a peripheral edge, loading channel and internal holding area, the internal holding area dimensioned to accept the largest medical wire the device is destined to be used with; and attachment means connected to a holding portion to secure the retaining device to a supporting surface; wherein a section of medical wire may be inserted into and along the loading channel to enter the holding area and be retained within.

This application claims the benefit of U.S. Provisional Patent Application No. 61/440,245, filed on Feb. 7, 2011, which is hereby incorporated by reference for all purposes as if fully set forth herein.

### BACKGROUND OF THE INVENTION

1. Field of the Invention

The invention relates to spiral wire retaining devices and, more specifically, spiral wire retaining devices for securing medical wires, catheters, guidewires, electrical lines, tubing, cables, and other elongated members.

2. Discussion of the Related Art

Guidewires and catheters are used in a variety of different medical procedures, such as, during angiographic, endovascular, or surgical procedures. Guidewires are typically used to position catheters in a body lumen, for example, arteries, veins or natural orifices within a mammal. The leading end portion of the guidewire is typically introduced into the body through an incision or natural orifice and then advanced to the treatment area. A catheter can be threaded over the guidewire, and advanced over the guidewire to the treatment area.

A single guidewire can be used to deliver multiple catheters to the treatment area within the body lumen. This is normally accomplished by withdrawing and removing the first catheter from the wire while leaving the leading edge of the wire in place within the body lumen, and then threading a second catheter over the wire and advancing down to the treatment site. Multiple guidewires and catheters can be inserted into the body lumen at the same time.

When the guidewire or a guidewire and catheter combination is placed within the body lumen, a loose trailing end portion extends out of the patient from the entry point. This trailing end portion can be difficult to manage and requires special attention from the medical staff to ensure it does not become contaminated, tangled or confused with other wires in the procedural area.

Therefore, a device and method for retaining the trailing end of guidewires and catheters is desired.

### SUMMARY OF THE INVENTION

Accordingly, this invention is directed towards a spiral wire retention device and method of using the same that substantially obviates one or more of the problems due to limitations and disadvantages of the related art.

An advantage of the invention is to provide an apparatus to securely retain a medical wire to a medical surface while providing ease of loading and removal of the medical wire from the apparatus.

Another advantage of the invention is to provide a simple and secure means for attaching the apparatus to a medical surface.

Another advantage of the invention is to provide a retention device that allows guidewire and catheter exchanges through it without having to remove the guidewire or catheter from the retention device.

Yet another advantage of the invention is to provide an apparatus that is configured to be easy to insert, remove, securely retain medical devices, and secure to the various surfaces encountered during medical procedures. Moreover, the apparatus is configured to allow for the insertion and withdrawal of catheters over retained guidewires during interventional procedures. Thereby, removing the guidewire from the retaining device during a catheter removal or insertion prevents additional risk that the guidewire might fall from the procedural area and become contaminated. It also allows for placement of the retaining device further from the catheter insertion point, thus adding more support where it is needed, near the end of the guidewire. In one embodiment, the spiral or helical holder structure also has advantages over other structures in that it has a low profile or high ratio of holder area to device diameter.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, an aspect of the invention is directed towards a retaining device for securing a medical apparatus during a medical procedure includes a base portion and a wire movably coupled to the base portion. The wire is arranged at a predetermined angle from the base portion having a peripheral edge including a slot extending from the peripheral edge defining a loading channel and communicating with an internal holding area. The internal holding area is dimensioned to accept the medical apparatus via the loading channel. Optionally, an attachment mechanism is configured to secure the base portion to a substrate.

Another aspect of the invention is directed towards a method of using a spiral wire retaining device to secure a medical device to a predetermined location. The method includes attaching the spiral wire retaining device to a substrate. The spiral wire retaining device includes a loading channel and a holding area. The method also includes arranging a portion of the medical device through the loading channel into a holding area of the spiral wire retaining device.

Yet another aspect of the invention is directed towards a medical kit including a retaining device according to any embodiment and instructions for use. The medical kit may include other devices that the retaining device is contemplated to be used with.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention, are incorporated in and constitute a part of this specification They illustrate embodiments of the invention and together with the description serve to explain the principles of the invention.

In the drawings:

FIG. 1 is a perspective view of a retaining device according to an embodiment of the invention;

FIG. 2 is a perspective view of a retaining device according to another embodiment of the invention;

FIG. 3 is a partial, perspective view of a sheet securing feature of the embodiment shown in FIG. 2;

FIG. 4 is a partial, perspective view of the sheet securing feature of the embodiment shown in FIG. 2;

FIG. 5 is an inferior, partial, perspective view of the sheet securing feature of the embodiment shown in FIG. 2;

FIG. 6 is a perspective view of another embodiment of the invention; and

FIG. 7 is a perspective view of another embodiment of the invention.

### DETAILED DESCRIPTION OF THE ILLUSTRATED EMBODIMENTS

An aspect of the invention is directed towards a spiral wire retaining device, or more specifically, towards a spiral wire retaining device for securing medical wires, catheters, guidewires, electrical lines, tubing, cables, and other elongated members. The expression “medical device” will be used herein to include all kinds of elongated members used during medical procedures, in particular catheters and guidewires.

In one embodiment, the retaining device includes a spiral-shaped holder portion defining a peripheral edge, loading channel and internal holding area. The internal holding area is dimensioned to accept the largest medical device that the retaining device is configured to be used with. An attachment mechanism is connected to the holder portion to secure the retaining device to a supporting surface, e.g., substrate, such as, a sheet, a table, gown and the like. A portion of medical device may be inserted into and along the loading channel to enter the holding area to be retained within.

In one embodiment, the holder portion is flexible and a portion of the loading channel is dimensioned to be smaller than the smallest medical device to be used with the retaining device. The medical device may enter through the channel and into the internal holding area via a mechanical force which causes flexure of the spiral structure, thereby permitting entry into the holding area.

In one embodiment, the spiral structure cross section may be configured as any geometric shape, e.g., round, rectangular, square, triangular, pentagon, hexagon, octagon, angular and/or combinations of the same. That is, there may be more than one shape, e.g., a plurality of cross sectional shapes in the same spiral structure.

In one embodiment, the spiral structure has varying cross sectional areas along its length.

In one embodiment, the ends of the spiral structure have a bent shape to facilitate insertion and removal of the medical wire from the retaining device.

In one embodiment, the holder portion is a helix or a tapered helix structure.

In one embodiment, the holder portion is connected to a plate shaped base portion with an adhesive coating for securing the retaining device to a supporting surface.

In one embodiment, the holder portion is connected to a plate shaped base portion with an integral feature for securing the device to a sheet or drape. The feature includes a tab having a geometric shape, such as, a square, rectangular or other shape, cut from the base portion. The tab may also include a barb or plurality of barbs to aid in adhesion to the supporting surface. Moreover, the securing mechanism may include a slot mechanism as described with reference towards U.S. Pat. No. 7,229,051, which is hereby incorporated by reference as if fully set forth herein.

In one embodiment, the holder portion is connected to a plate shaped base portion with an attachment feature that allows the spiral structure to be folded nearly parallel to the base portion. The structure may also be configured at an angle from about 5 degrees to about 175 degrees, more preferably in a range from about 45 degrees to about 135 degrees and most preferably from about 75 degrees to about 115 degrees. In another embodiment the angle is about 90 degrees.

In one embodiment, the retaining device is a one-piece structure with an integral sheet locking feature composed of wire bent into at least one elongated geometric shape, e.g., a paperclip type shape.

In one embodiment, a retaining device for securing a medical apparatus during a medical procedure includes a base portion and a wire movably coupled to the base portion. The wire is arranged at a predetermined angle from the base portion having a peripheral edge including a slot extending from the peripheral edge defining a loading channel and communicating with an internal holding area. The internal holding area is dimensioned to accept the medical apparatus via the loading channel. Optionally, an attachment mechanism is configured to secure the base portion to a substrate.

In one embodiment, the invention is directed towards a method of using a spiral wire retaining device to secure a medical device to a predetermined location. The method includes attaching the spiral wire retaining device to a substrate. The spiral wire retaining device includes a loading channel and a holding area. The method also includes arranging a portion of the medical device through the loading channel into a holding area of the spiral wire retaining device. In one embodiment, a medical kit includes a retaining device according to any embodiment and instructions for use. The medical kit may include other devices that the retaining is contemplated to be used with.

Reference will now be made in detail to an embodiment of the present invention, example of which is illustrated in the accompanying drawings.

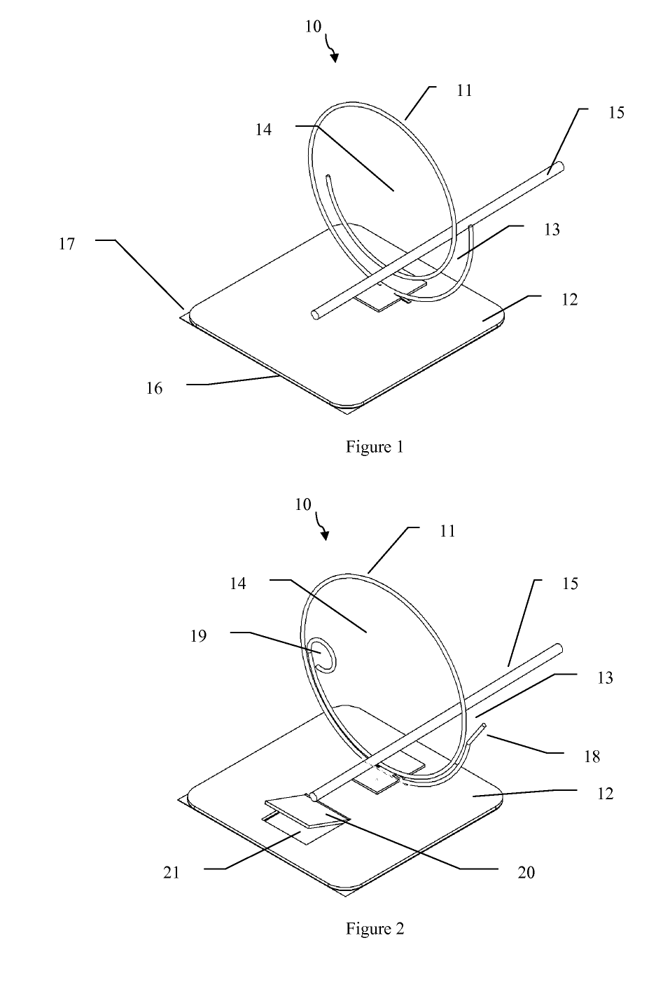

FIG. 1 illustrates a retaining device 10 according to an embodiment of invention. In the embodiments shown in the drawings, medical device retaining device may be in any geometric configuration, e.g., a spiral-shaped holding portion 11 attached to a base portion 12. Base structure 12 may be configured as rectangular plate shaped structure and forms the member of the medical device support retaining device 10 will rest upon. Base portion 12 attaches to the spiral holding portion 11 near the outer-most periphery of the spiral-shaped holding portion 11. An attachment mechanism includes at least one of an adhesive, adhesive tape, rivets, brackets, holes, slots and/or combinations of the same. The attachment mechanism allows the spiral holding portion 11 to fold down parallel to the base portion 12. Spiral holder 11 may be placed at any angle, in a preferred embodiment, at a right angle to base 12 during use. The ability to change this angle and fold it flat is advantageous, for example, for product packaging, during the procedure when device in not in use and further other desired angles that may be advantageous during a procedure.

In this embodiment, spiral holding portion 11 includes a wire configured in a spiral shape having a predetermined diameter, pitch, and number of rotations to define a loading channel 13 and holding area 14. In use, a portion of medical device 15 is inserted in loading channel 13 at the outer-most periphery of the spiral and then slid along the channel till it drops into the holding area 14. Once in the holding area 14 the medical device is retained. To remove the medical device 15, the device must be moved to the channel 13 at the innermost periphery and slid along the channel 13 to the outermost periphery of the spiral holding portion 11.

Referring to FIG. 1, the pitch of the spiral defining the width of the channel 13 is designed to accept the largest size medical device and configured so that medical device insertion or removal has no resistance. This embodiment relies on gravity to retain the medical wire in the holder area 14. One advantage of the spiral shape is that it can be designed with multiple securing features. For example, if medical device 15 within holder area 14 accidentally found the channel 13 and dropped down the channel to the base 12 it would still remain secured. Increasing the number of rotations of the spiral would increase this safety feature.

In addition, the holding area 14 can be sized to accept the largest medical device and also allow any attachments to the medical device to pass completely through the working area 14. For example, a typical catheter will have a shaft diameter of 1.5 millimeters (mm), however the trailing end of the catheter may have a fluid connection fitting with a width of 10 mm and connected to the fitting such as a hemostasis valve with a width of 5 centimeters (cm). Thus in this example the holding area 14 would be dimensioned to allow the 5 cm valve to pass completely through. In one embodiment, the holding area would have a size in a range from about 0.05 inches² to about 28 inches², a preferred range from about 0.2 inches² to about 20 inches², and a most preferred range from about 0.7 inches² to about 7 inches².

In FIG. 1, base 12 contains an adhesive securing mechanism 16 for connecting the base 12 to a support structure (not shown). A typical securing mechanism includes a pressure sensitive adhesive covering at least a portion of the bottom of the base portion 12. In a preferred embodiment, an adhesive 16 is covered with a protective, peel-off backing 17 until use. Other types of securing mechanisms could also be used. For example, base could be a heavy, draping pad such as a mouse pad. Mouse pads are typically made with flexible, heavy polymers such as rubber, neoprene rubber, silicone, and silicone foam. A mouse pad holds to a surface without sliding due to weight, a large contacting surface area, a textured contacting surface area, and high coefficient of friction of the before mentioned materials. FIG. 1 shows an embodiment of the medical wire retaining device 10 where the spiral holding portion 11 has a round cross-section. The cross-section shape of the holding portion may be configured into any desired geometric configuration, e.g., a rectangular, triangular, square, and/or combinations of the same. In addition, the cross-section shape may vary along its length. Finally, there may be multiple holding portions, e.g., there may be two or more holding portions each configured to accept a medical device.

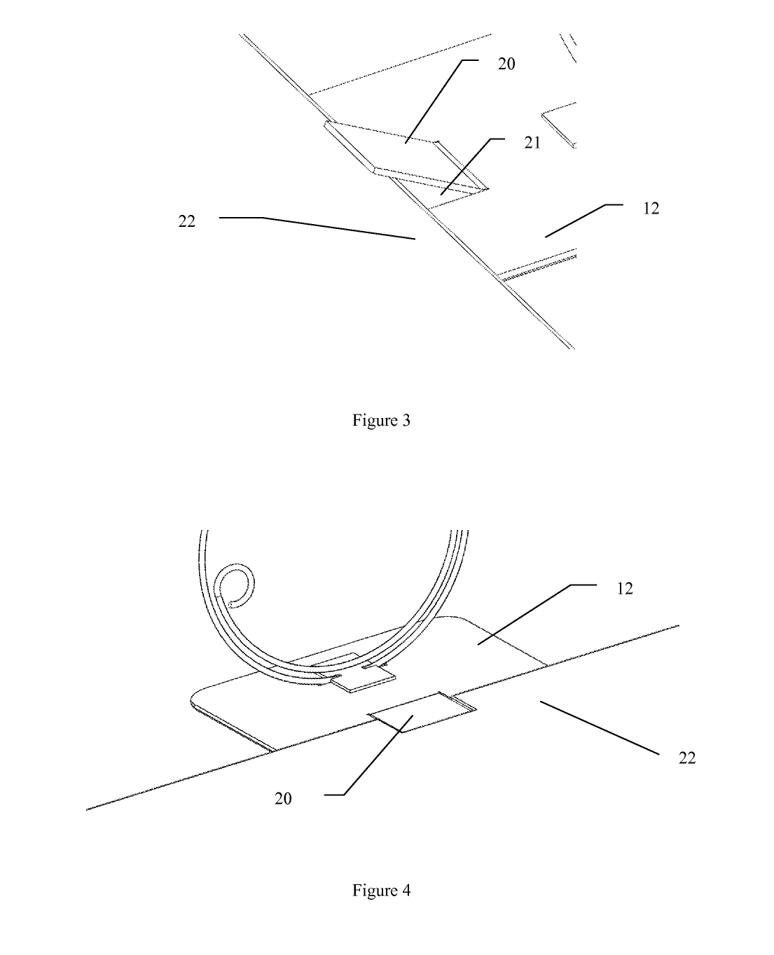

FIG. 2 shows another embodiment of the invention. Referring to FIG. 2, the spiral pitch has been modified to create a channel width of less than the smallest medical device 15 configured to be used with the device, e.g., a width of about 0 inches or less to about 1 inch or greater. This embodiment adds an extra feature for securing the medical wire in the holding area 14. The feature is frictional resistance of the channel 13. In order for the medical device 15 to move through the channel 13 the spiral must flex and allow the channel to open and accept the diameter of the medical device 15. The flex of the spiral holding portion 11 is controlled by the cross sectional area, spiral diameter, modulus of the wire material and diameter of the wire. Furthermore, a first bend 18 and second bend 19 have been added to the ends of the spiral holding portion 11 to facilitate insertion and removal of the medical device 15. A straight, angular bend is shown in bend 18 and a circular bend is shown in bend 19. However, other geometric configurations may also be used, for example, the ends may be spiral shaped, simply bent over to produce a radiused end, formed to a solid ball shaped end, and combinations of the same.

Also shown in FIG. 2 is yet another embodiment for securing retaining device 10 to a substrate, e.g., a bed sheet, a medical drape, gown and the like. Base 12 has an integral rectangular or square tab 20 that has been cut from the base. Tab 20 has three sides and a bend on the remaining side connected to base 12 and angling up from the base. Below the bent tab is a rectangular or square orifice 21 in the base approximately the same size as the tab. Any geometrical shape of the tab may be utilized, e.g., triangle, semi-circle, multiple peaks, and the like. Moreover, the tab 20 may include protrusions, e.g., barbs, to aid in securing to the bed sheet, drape or gown. Another securing mechanism may include a slot dimensioned as a compressive fit as described with reference to U.S. Pat. No. 7,229,051, which is hereby incorporated by reference as if fully set forth herein. In addition, other securing mechanisms may be utilized, for example, a securing device as described in U.S. Pat. No. 1,389,304, which is hereby incorporated by reference as if fully set forth herein.

FIG. 3 shows an edge or folded edge of a sheet 22 placed over the base 12 and under the tab 20. To secure the retaining device 10 to the sheet 22, the tab is pushed down and through the orifice 21 and slightly below the plane of the base, wedging the sheet 22 between the tab 20 and orifice 21.

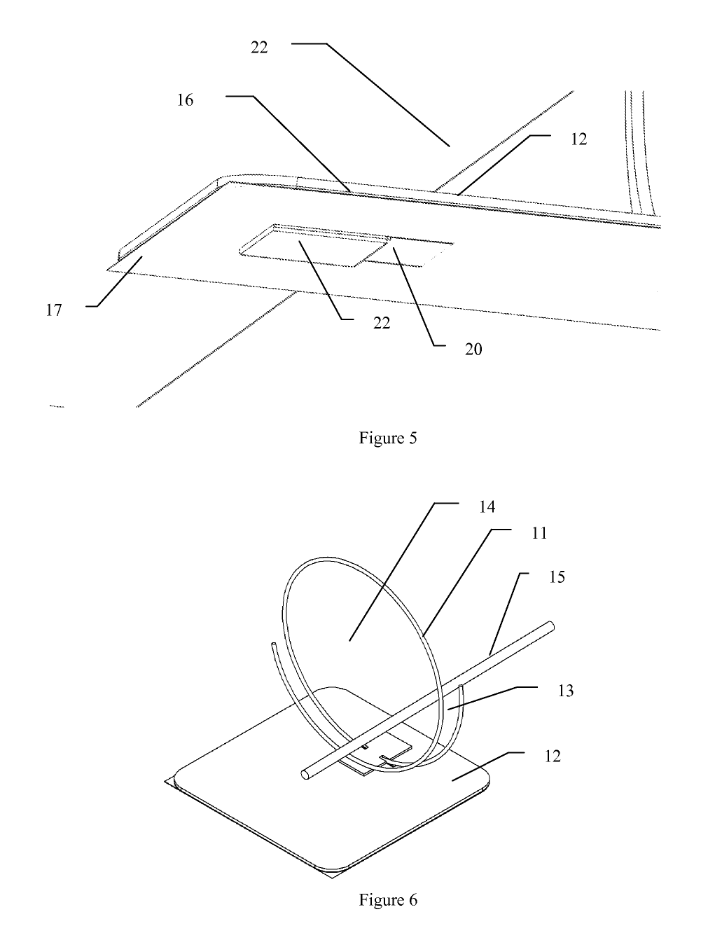

FIGS. 4 and 5 show the sheet 22 in the locked position as viewed from above and below. This embodiment provides a simple, securing mechanism for securing the retaining device to a substrate, e.g., sheet, drape, gown and the like. To disengage the sheet 22, push the tab 20 back out the other direction. As shown in FIG. 5, an advantage of this securing mechanism is that it can be combined with the adhesive 16 to provide increased utility to secure to many different types of substrates. Moreover, a barb or plurality of barbs may also be used to aid in adhesion to a substrate.

FIG. 6 illustrates another embodiment of the invention. Referring to FIG. 6, the holding portion 11 is a tapered helix structure having a peripheral edge, loading channel 13 and internal holding area 14. It is similar to the spiral structure described in previous embodiments with the exception that the taper of the helix defines the width of the loading channel 13.

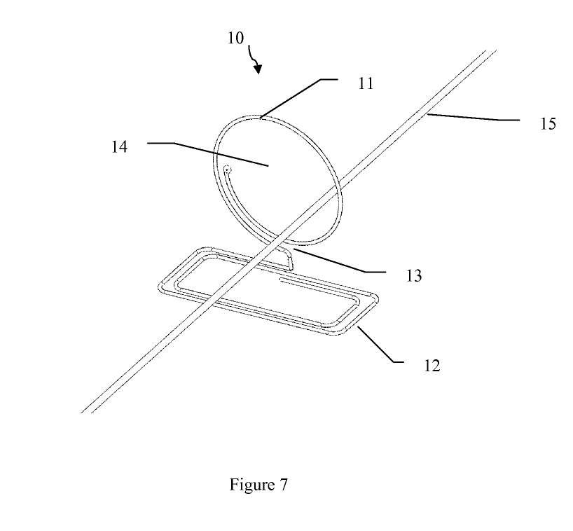

FIG. 7 illustrates another embodiment of the invention. Referring to FIG. 7, the medical wire retaining device 10 includes a one-piece structure, e.g., wire. In this embodiment, the holding portion 11 is a spiral shaped structure having a peripheral edge, loading channel 13 and internal holding area 14. At the bottom periphery of the holding portion 11, the spiral wire is bent to form a rectangular base portion 12. Base portion 12 includes a wire formed in a rectangular spiral shape consisting of 2 full revolutions. The base portion 12 can be attached to substrate, e.g., a bed sheet, medical drape or gown. To secure, the edge or fold of the sheet is inserted between the outer and inner revolutions of the wire like a paper-clip. Of course, one or more other geometric configurations may also be used as described herein.

### EXAMPLES

Without intending to limit the scope of the invention, the following examples illustrate how various embodiments of invention may be made and/or used.

### Example 1

In this example, a spiral holder portion was fabricated to the shape similar to that shown in FIG. 2. A stainless steel wire having a 0.045 diameter, a 304 spring temper was formed with a CNC machine. The inner most diameter is about 2.5 inches and the spiral winds to the outside with a 0.14 pitch for 1.4 revolutions. The inner most terminal end of the wire is formed to about 0.4 inches in diameter circle and 0.5 inches of the outer most terminal end is bent outwardly 20 degrees as shown in FIG. 2.

The base was formed by cutting a 0.125 inch thick neoprene pad to a 5×7 inch shape. A bracket for attaching spiral holder portion to base was formed by cutting a 0.04 thick polyethylene terephthalate glycol (PETG) sheet material to about 1.4×1.4 inch in size. Two slots, measuring about 0.05 inch wide by 0.25 inch long are cut in the center of two opposite sides of the bracket as shown in FIG. 2. The bracket was placed along the outermost spiral between the terminal ends of the spiral so that the wire fits within the slots as shown in FIG. 2. The bracket was then adhered to the base using hot melt adhesive, securing the spiral holder portion between the base and bracket.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A retaining device for securing a medical apparatus during a medical procedure, comprising:
a base portion;
a wire movably coupled to the base portion, the wire arranged at a predetermined angle from the base portion having a peripheral edge including a slot extending from the peripheral edge defining a loading channel and communicating with an internal holding area, wherein the internal holding area is dimensioned to accept the medical apparatus via the loading channel; and
an attachment mechanism configured to secure the base portion to a substrate.

2. The apparatus of claim 1, wherein the predetermined angle is a range from about 30 degrees to about 170 degrees.

3. The apparatus of claim 1, wherein the medical apparatus comprises at least one of a medical wire, a guide wire, a catheter, an electrical line, a cable, and a tubing.

4. The apparatus of claim 1, wherein the predetermined angle is about 90 degrees.

5. The apparatus of claim 1, wherein the wire is configured into a spiral geometric shape.

6. The apparatus of claim 1, wherein the attachment mechanism comprises an adhesive.

7. The apparatus of claim 1, wherein the attachment mechanism is formed in the base portion and comprises a tab and an orifice configured to receive a portion of the supporting substrate.

8. The apparatus of claim 7, wherein the tab comprises at least three sides that correspond to the orifice.

9. The apparatus of claim 8, wherein the tab comprises at least one barb.

10. The apparatus of claim 1, wherein the internal holding area is configured into two or more holding areas.

11. The apparatus of claim 1, wherein the wire comprises a diameter in a range from about 0.01 inches to about 0.2 inches.

12. The apparatus of claim 1, wherein the wire comprises a material selected from a group consisting of steel, stainless steel, nitinol, titanium, aluminum, nylon, polycarbonate, polysulfonone, polyether ether ketone (PEEK), acrylonitrile butadiene styrene (ABS), polyethylene terephthalate (PET), polyethylene terephthalate glycol (PETG) and combinations of the same.

13. The apparatus of claim 1, wherein the portion of the loading channel width is dimensioned to be smaller than the portion of the medical apparatus.

14. The apparatus of claim 1, wherein the supporting substrate comprises a drape.

15. The apparatus of claim 1, wherein the attachment mechanism comprises a polymer pad with at least a surface area of about 15 inches² or greater.

16. The apparatus of claim 5, wherein the base portion comprises the wire.

17. The apparatus of claim 5, wherein the spiral geometry comprises at least two loops.

18. A method of using a spiral wire retaining device to secure a medical device to a predetermined location, comprising the steps of:
attaching the spiral wire retaining device to a substrate, wherein the spiral wire retaining device comprises a loading channel and a holding area; and
arranging a portion of the medical device through the loading channel into a holding area of the spiral wire retaining device.

19. The method of claim 18, wherein the medical device comprises at least one of a medical wire, a guide wire, a catheter, an electrical line, a cable, and a tubing.

20. A medical kit, comprising:
a spiral wire retaining device, comprising:
a base portion; and
a spiral wire coupled to and arranged at a predetermined angle from the base portion having a peripheral edge including a slot extending from the peripheral edge defining a loading channel and communicating with an internal holding area, wherein the internal holding area is dimensioned to accept the medical device; and
instructions for use.
